(19) United States
(12) Patent Application Publication
(10) Pub. No.: US 2026/0356885 A1
(43) Pub. Date: Nov. 12, 2026

(54) PROCESS FOR MANUFACTURING AN ANTIBIOTIC MACROCYCLIC PEPTIDE

(71) Applicant: Hoffmann-La Roche Inc., Little Falls, NJ (US)

(72) Inventors: Raphael BIGLER, Frauenfeld (CH); Fritz Theodor BLISS, Hartheim (DE); Serena Maria FANTASIA, Saint-Louis (FR); Fabienne Roxane HOFFMANN-EMERY, Delémont (CH); Ernesto SANTANDREA, Zürich (CH)

(73) Assignee: Hoffmann-La Roche Inc., Little Falls, NJ (US)

(21) Appl. No.: 19/102,353

(22) PCT Filed: Aug. 7, 2023
(86) PCT No.: PCT/EP2023/071759

§ 371 Date: Feb. 7, 2025

(30) Foreign Application Priority Data
Aug. 9, 2022 (EP) 22189412.4

(51) Int. Cl. C07K 5/12; C07K 1/02; C07K 1/107; C07K 1/30

(52) U.S. Cl. CPC C07K 5/123; C07K 1/02; C07K 1/1077; C07K 1/306

(57) ABSTRACT

The invention relates to a novel process for manufacturing 4-[(11S, 14S, 17S)-14-(4-Aminobutyl)-11-(3-aminopropyl)-17-(1H-indol-3-ylmethyl)-16-methyl-12, 15,18-trioxo-2-thia-4,10, 13, 16, 19-pentazatricyclo [19.4.0.03,8]pentacosa-1 (25),3(8),4,6,21,23-hexaen-22-yl]benzoic acid (I), or a pharmaceutically acceptable salt thereof. The invention further relates to certain synthetic intermediates that are useful for the novel process according to the invention. The process according to the invention is particularly suitable for large-scale manufacturing of the compound of formula (I) under GMP conditions.

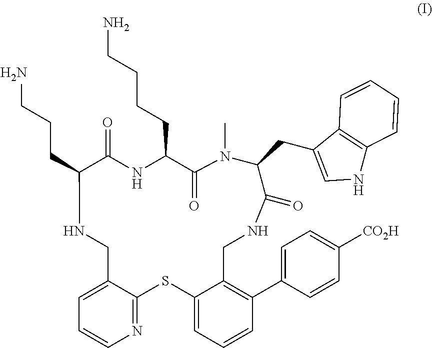 [SMILES: CN1C(=O)[C@H](CCCCN)NC(=O)[C@H](CCCN)NCc2cccnc2Sc2cccc(-c3ccc(C(=O)O)cc3)c2CNC(=O)[C@@H]1Cc1c[nH]c2ccccc12]

### FIELD OF THE INVENTION

The invention relates to a novel process for manufacturing 4-[(11S,14S,17S)-14-(4-Aminobutyl)-11-(3-aminopropyl)-17-(1H-indol-3-ylmethyl)-16-methyl-12,15,18-trioxo-2-thia-4, 10, 13, 16, 19-pentazatricyclo[19.4.0.03,8]pentacosa-1 (25),3 (8), 4,6,21,23-hexaen-22-yl]benzoic acid (I), or a pharmaceutically acceptable salt thereof.

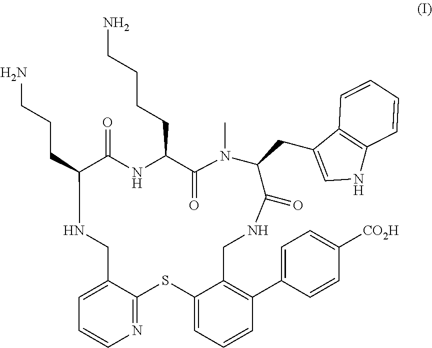 [SMILES: CN1C(=O)[C@H](CCCCN)NC(=O)[C@H](CCCN)NCc2cccnc2Sc2cccc(-c3ccc(C(=O)O)cc3)c2CNC(=O)[C@@H]1Cc1c[nH]c2ccccc12]

The invention further relates to certain synthetic intermediates that are useful for the novel process according to the invention.

The process according to the invention is particularly suitable for large-scale manufacturing of the compound of formula (I) under GMP conditions.

### BACKGROUND OF THE INVENTION

The compound of formula (I) is a potent antibiotic with selective action against Acinetobacter baumannii, as discussed in WO2019206853. The compound of formula (I) is also known under the INN zosurabalpin (WHO Drug Information, Vol. 36, No. 2, 2022).

WO2019206853 discloses a laboratory scale synthesis of the compound of formula (I), which relies on a Suzuki coupling reaction that is characterized by a high catalyst loading (about 20 mol %) and a relatively high amount of a boronic acid building block (about 1.5 equivalents). The high catalyst loading, in addition to generating high costs, also leads to palladium impurities in the final product that are difficult to remove. Similarly, the use of high amounts of boronic acid results in the formation of organic impurities. Furthermore, the process disclosed in WO2019206853 involves the use of the toxic solvent dioxane, and relies on chromatography to purify certain intermediates, as well as the final product.

In summary, the process for making the compound of formula (I) as described in WO2019206853 is not well suited for an industrial scale synthesis of said compound of formula (I). Accordingly, there is a high unmet need for a new process for manufacturing the compound of formula (I), in order to provide patients with this new treatment option for infections with Acinetobacter baumannii and resulting diseases.

### SUMMARY OF THE INVENTION

The present invention provides an improved process for manufacturing the compound of formula (I), which overcomes the problems outlined above. The present invention also provides certain synthetic intermediates that are useful in the new process.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims and the abstract), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims and the abstract), or to any novel one, or any novel combination, of the steps of any method or process so disclosed.

The term “palladium catalyst” as used herein refers to any palladium catalyst that enables the reaction of aryl bromide 1 as described herein with (4-carbomethoxyphenyl) boronic acid (2) to afford compound 3 as defined herein on an industrial scale. The palladium catalyzed reaction according to the invention requires a zero valent palladium species (Pd(0)). Exemplary catalytically active Pd(0) species may be applied directly or may be formed in situ from a palladium source in combination with a phosphine ligand. In some embodiments, the palladium catalyst is a preformed palladium catalyst. Examples of preformed catalytically active Pd(0) reagents include: Pd(PPh₃)₄ and Pd(L)₂, wherein L is selected from PBu₃, AmPhos, CPhos, RuPhos, and SPhos. Further examples of preformed palladium catalysts include: [Pd(L)XCl] (L=ligand as defined above, X=allyl, 2-butenyl, 2-methylallyl, 1-phenylallyl, p-tert-butylindenyl); [Pd(L)X]trifluoromethanesulfonate (L=ligand as defined above, X=as defined above), [Pd(L)(2-(2′-amino-1,1′-biphenyl) Cl] (L=as defined above); [Pd(L)(2-(2′-amino-1,1′-biphenyl)]methanesulfonate (L=as defined above), [Pd(L)(2-(2′-methylamino-1,1′-biphenyl)]methanesulfonate (L=as defined above), [Pd(L)(2-(2-aminoethyl)phenyl)Cl] (L=as defined above), PdLCl₂, PdL₂Cl₂ (L=PtBu₃, AmPhos, PtBu₂Bu, PtBu₂Ph, PtBu₂-p-C₆H₄CF₃)₂, dppf, dcypf, dippf, dtbpf. [PdI(L)]₂ (L=PtBu₃, AmPhos). Examples of palladium sources that form a palladium catalyst in situ in combination with a suitable ligand include: palladium bis(dibenzylideneacetone)(Pd(dba)₂), (Pd₂(dba)₃), bis(triphenylphosphine) palladium (II) dichloride (Pd(PPh₃)₂Cl₂), palladium acetate (Pd(OAc)₂, palladium trifluoracetate (Pd(TFA)₂), palladium chloride (PdCl₂), palladium bromide (PdBr₂),palladium iodide (PdI₂), palladium bis acetylacetonate (Pd(acac)₂), (Pd(PPh₃)₄), bis(acetonitrile)-palladium(II) dichloride (PdCl₂(CH₃CN)₂), cyclopentadienyl allyl palladium, allylpalladium(II) chloride dimer (Pd(allyl)Cl)₂), (2-butenyl)chloropalladium dimer, (2-methylallyl) palladium(II) chloride dimer, palladium (1-phenylallyl) chloride dimer, (p-tert-butylindenyl) palladium(II) chloride dimer, di-μ-chlorobis [2′-(amino-N)[1,1′-biphenyl]-2-yl-C]dipalladium(II), di-μ-mesylbis[2′-(amino-N) [1,1′-biphenyl]-2-yl-C]dipalladium(II), di-μ-mesylbis[2′-(methylamino-N)[1,1′-biphenyl]-2-yl-C]dipalladium(II), and di-μ-chlorobis[2-[(dimethylamino)methyl]phenyl-C,N]dipalladium(II). Examples of ligands that form palladium catalysts in situ in combination with a palladium source described above include: mono or diphosphines such as PtBu₃, PAd₃, AmPhos, cataCXiumA, PtBu₂Bu, PtBu₂Ph, PtBu₂-p-C₆H₄CF₃, cataCXium POMeCy, CPhos, RuPhos, SPhos, dppf, dcypf, dippf, and dtbpf.

The term “salt” as used herein refers to any kind of salts formed by reacting the compounds disclosed herein with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. Where the compounds disclose herein contain a free acidic moiety, salts may also be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

### Manufacturing Process

In a first aspect, the present invention provides a process for manufacturing compound 3, or a salt thereof,

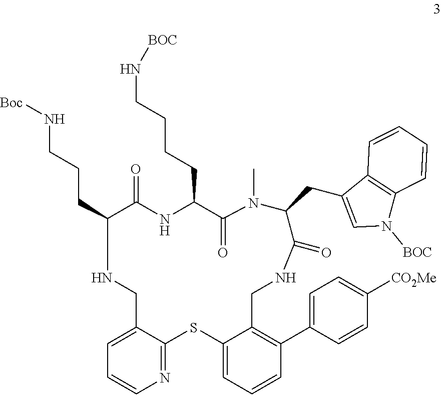

comprising:
(a) reacting aryl bromide 1

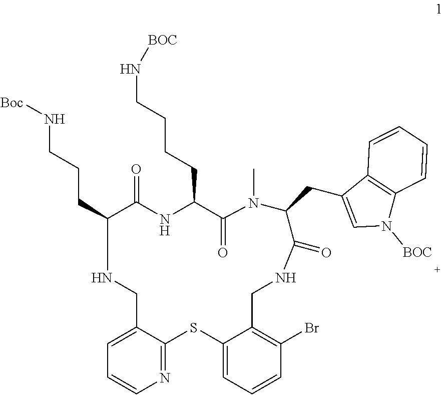

with (4-carbomethoxyphenyl) boronic acid (2)

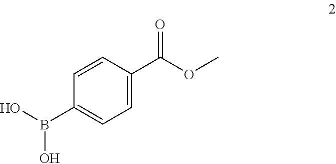

in the presence of a palladium catalyst and a base, to afford said compound 3.

In a further aspect, the present invention provides a process for manufacturing compound 3, or a salt thereof,

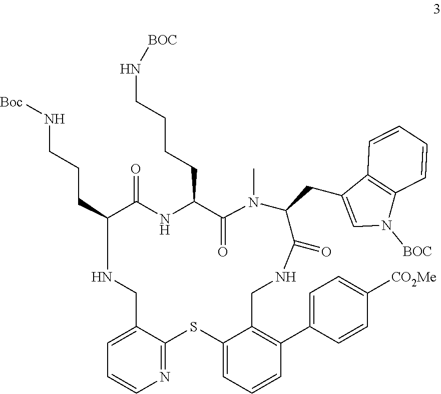

comprising:
(a) reacting aryl bromide 1

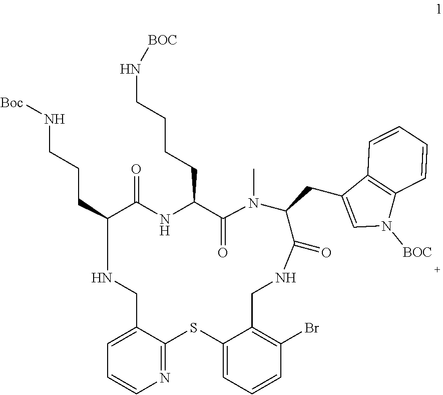

with (4-carbomethoxyphenyl) boronic acid (2)

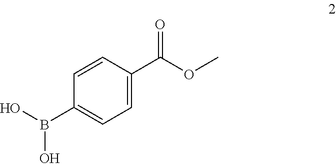

in the presence of a palladium catalyst and a base in an aromatic solvent, to afford said compound 3; wherein said palladium catalyst is selected from:
i) a preformed catalyst selected from Pd(PPh₃)₄, Pd(L¹)₂, [Pd(L¹)XCl], [Pd(L¹)X]trifluoromethanesulfonate, [Pd(L¹)(2-(2′-amino-1,1′-biphenyl)Cl]; [Pd(L¹)(2-(2′-amino-1,1′-biphenyl)]methanesulfonate, [Pd(L¹)(2-(2′-methylamino-1,1′-biphenyl)]methanesulfonate, [Pd(L¹)(2-(2-aminoethyl)phenyl)Cl], Pd(L²)Cl₂, Pd(L²)₂Cl₂, and [PdI(L³)]₂; wherein X is selected from allyl, 2-butenyl, 2-methylallyl, 1-phenylallyl, p-tert-butylindenyl; L¹ is selected from PBu₃, AmPhos, CPhos, RuPhos, and SPhos; L² is selected from PtBu₃, AmPhos, PtBu₂Bu, PtBu₂Ph, PtBu₂-p-C₆H₄CF₃)₂, dppf, dcypf, dippf, and dtbpf; and L³ is selected from PtBu₃, AmPhos; and
ii) a catalyst that is formed in situ from:
   iia) a palladium source selected from palladium bis(dibenzylideneacetone) (Pd(dba)₂), (Pd₂(dba)₃), bis(triphenylphosphine) palladium (II) dichloride (Pd(PPh₃)₂Cl₂), palladium acetate (Pd(OAc)₂, palladium trifluoracetate (Pd(TFA)₂), palladium chloride (PdCl₂), palladium bromide (PdBr₂),palladium iodide (PdI₂), palladium bis acetylacetonate (Pd(acac)₂), (Pd(PPh₃)₄), bis(acetonitrile)-palladium (II) dichloride (PdCl₂ (CH₃CN)₂), cyclopentadienyl allyl palladium, allylpalladium (II) chloride dimer (Pd(allyl)Cl)₂), (2-butenyl) chloropalladium dimer, (2-methylallyl) palladium (II) chloride dimer, palladium (1-phenylallyl) chloride dimer, (p-tert-butylindenyl) palladium (II) chloride dimer, di-μ-chlorobis[2′-(amino-N)[1,1′-biphenyl]-2-yl-C]dipalladium(II), di-μ-mesylbis[2′-(amino-N)[1,1′-biphenyl]-2-yl-C]dipalladium(II), di-μ-mesylbis[2′-(methylamino-N)[1,1′-biphenyl]-2-yl-C]dipalladium(II), and di-μ-chlorobis [2-[(dimethylamino)methyl]phenyl-C,N]dipalladium(II); and
   iib) a ligand selected from mono- and diphosphines, in particular a ligand selected from PtBu₃, PAd₃, AmPhos, cataCXiumA, PtBu₂Bu, PtBu₂Ph, PtBu₂-p-C₆H₄CF₃, cataCXium POMeCy, CPhos, RuPhos, SPhos, dppf, dcypf, dippf, and dtbpf, and
said base is selected from inorganic bases and alcoholates, preferably wherein said base is selected from Na₂CO₃, Ba(OH)₂, K₃PO₄, Cs₂CO₃, K₂CO₃, TIOH, KF, CsF, Bu₄F, and NaOH, more preferably wherein said base is K₃PO₄ or K₂CO₃.

Suzuki coupling of aryl bromide 1 with (4-carbomethoxyphenyl) boronic acid (2) instead of with (4-(tert-Butoxycarbonyl)phenyl) boronic acid as described in WO2019206853 affords the new intermediate 3, which can be partially deprotected under basic conditions to afford new intermediate 4 (vide infra). This is in contrast to the gobal deprotection under acidic conditions (trifluoroacetic acid) described in WO2019206853. Importantly, new intermediate 4 is crystalline and can thus be conveniently purified by crystallization, replacing the need for chromatography as described in WO2019206853.

In one embodiment of step (a) of the process according to the invention, said palladium catalyst is selected from:
i) a preformed catalyst selected from Pd(PPh₃)₄, Pd(L¹)₂, [Pd(L¹)XCl], [Pd(L¹)X]trifluoromethanesulfonate, [Pd(L¹)(2-(2′-amino-1,1′-biphenyl)Cl]; [Pd(L¹)(2-(2′-amino-1,1′-biphenyl)]methanesulfonate, [Pd(L¹)(2-(2′-methylamino-1,1′-biphenyl)]methanesulfonate, [Pd(L¹)(2-(2-aminoethyl)phenyl)Cl], Pd(L²)Cl₂, Pd(L²)₂Cl₂, and [PdI(L³)]₂; wherein X is selected from allyl, 2-butenyl, 2-methylallyl, 1-phenylallyl, p-tert-butylindenyl; L¹ is selected from PBu₃, AmPhos, CPhos, RuPhos, and SPhos; L² is selected from PtBu₃, AmPhos, PtBu₂Bu, PtBu₂Ph, PtBu₂-p-C₆H₄CF₃)₂, dppf, dcypf, dippf, and dtbpf; and L³ is selected from PtBu₃, AmPhos; and
ii) a catalyst that is formed in situ from:
   iia) a palladium source selected from palladium bis(dibenzylideneacetone) (Pd(dba)₂), (Pd₂(dba)₃), bis(triphenylphosphine) palladium (II) dichloride (Pd(PPh₃)₂Cl₂), palladium acetate (Pd(OAc)₂, palladium trifluoracetate (Pd(TFA)₂), palladium chloride (PdCl₂), palladium bromide (PdBr₂), palladium iodide (PdI₂), palladium bis acetylacetonate (Pd(acac)₂), (Pd(PPh₃)₄), bis(acetonitrile)-palladium (II) dichloride (PdCl₂ (CH₃CN)₂), cyclopentadienyl allyl palladium, allylpalladium (II) chloride dimer (Pd(allyl)Cl)₂), (2-butenyl) chloropalladium dimer, (2-methylallyl) palladium (II) chloride dimer, palladium (1-phenylallyl) chloride dimer, (p-tert-butylindenyl) palladium (II) chloride dimer, di-μ-chlorobis [2′-(amino-N) [1,1′-biphenyl]-2-yl-C]dipalladium(II), di-μ-mesylbis [2′-(amino-N) [1,1′-biphenyl]-2-yl-C]dipalladium(II), di-μ-mesylbis [2′-(methylamino-N) [1,1′-biphenyl]-2-yl-C]dipalladium(II), and di-μ-chlorobis [2-[(dimethylamino)methyl]phenyl-C,N]dipalladium(II); and
   iib) a ligand selected from mono- and diphosphines, in particular a ligand selected from PtBu₃, PAd₃, AmPhos, cataCXiumA, PtBu₂Bu, PtBu₂Ph, PtBu₂-p-C₆H₄CF₃, cataCXium POMeCy, CPhos, RuPhos, SPhos, dppf, dcypf, dippf, and dtbpf.

In one embodiment of step (a) of the process according to the invention, said palladium catalyst is selected from Pd(PPh₃)₄, Pd(L¹)₂, [Pd(L¹)XCl],

[Pd(L¹)X]trifluoromethanesulfonate, [Pd(L¹)(2-(2′-amino-1,1′-biphenyl)Cl]; [Pd(L¹)(2-(2′-amino-1,1′-biphenyl)]methanesulfonate, [Pd(L¹)(2-(2′-methylamino-1,1′-biphenyl)]methanesulfonate, [Pd(L¹)(2-(2-aminoethyl)phenyl) Cl], Pd(L²)Cl₂, Pd(L²)₂Cl₂, and [PdI(L³)]₂; wherein X is selected from allyl, 2-butenyl, 2-methylallyl, 1-phenylallyl, p-tert-butylindenyl; L¹ is selected from PBu₃, AmPhos, CPhos, RuPhos, and SPhos; L² is selected from PtBu₃, AmPhos, PtBu₂Bu, PtBu₂Ph, PtBu₂-p-C₆H₄CF₃)₂, dppf, dcypf, dippf, and dtbpf; and L³ is selected from PtBu₃, AmPhos.

In one embodiment of step (a) of the process according to the invention, said palladium catalyst is formed in situ from:
i) a palladium source selected from palladium bis(dibenzylideneacetone)(Pd(dba)₂), (Pd₂(dba)₃), bis(triphenylphosphine) palladium (II) dichloride (Pd(PPh₃)₂Cl₂), palladium acetate (Pd(OAc)₂, palladium trifluoracetate (Pd(TFA)₂), palladium chloride (PdCl₂), palladium bromide (PdBr₂),palladium iodide (PdI₂), palladium bis acetylacetonate (Pd(acac)₂), (Pd(PPh₃)₄), bis(acetonitrile)-palladium (II) dichloride (PdCl₂(CH₃CN)₂), cyclopentadienyl allyl palladium, allylpalladium (II) chloride dimer (Pd(allyl)Cl)₂), (2-butenyl) chloropalladium dimer, (2-methylallyl) palladium (II) chloride dimer, palladium (1-phenylallyl) chloride dimer, (p-tert-butylindenyl) palladium (II) chloride dimer, di-μ-chlorobis [2′-(amino-N) [1,1′-biphenyl]-2-yl-C]dipalladium(II), di-μ-mesylbis [2′-(amino-N) [1,1′-biphenyl]-2-yl-C]dipalladium(II), di-μ-mesylbis [2′-(methylamino-N) [1,1′-biphenyl]-2-yl-C]dipalladium(II), and di-μ-chlorobis [2-[(dimethylamino)methyl]phenyl-C,N]dipalladium(II); and
ii) a ligand selected from mono- and diphosphines.

In a preferred embodiment, said mono- and diphosphine ligands are selected from PtBu₃, PAd₃, AmPhos, cataCXiumA, PtBu₂Bu, PtBu₂Ph, PtBu₂-p-C₆H₄CF₃, cataCXium POMeCy, CPhos, RuPhos, SPhos, dppf, dcypf, dippf, and dtbpf.

In a preferred embodiment of step (a) of the process according to the invention, said palladium catalyst is selected from Pd(PPh₃)₄, CPhosPdG3, Pd₂(dba)₃+CPhos, and PdCl₂(dtbpf).

It has been found that the palladium catalyst PdCl₂(dtbpf) works particularly well in the Suzuki coupling according to the invention. Thus, the Suzuki coupling with PdCl₂(dtbpf) requires only a very low catalyst loading (2 mol % or less) and a small excess of boronic acid 2, which results in reduced amount of contamination of the final product with palladium and organic impurities, reduced cost and reduced waste. Those are all important aspects when manufacturing chemicals on an industrial scale, especially when manufacturing pharmaceutical products.

Thus, in a particularly preferred embodiment of step (a) of the process according to the invention, said palladium catalyst is PdCl₂(dtbpf).

In one embodiment of step (a) of the process according to the invention, said palladium catalyst is present in an amount of 1 mol % to 10 mol % relative to aryl bromide 1.

In one embodiment of step (a) of the process according to the invention, said palladium catalyst is present in an amount of 1 mol % to 5 mol % relative to aryl bromide 1.

In a preferred embodiment of step (a) of the process according to the invention, said palladium catalyst is present in an amount of 1 mol % to 3 mol % relative to aryl bromide 1.

In a particularly preferred embodiment of step (a) of the process according to the invention, said palladium catalyst is present in an amount of 2 mol % relative to aryl bromide 1.

In a preferred embodiment of step (a) of the process according to the invention, said palladium catalyst is PdCl₂(dtbpf) and is present in an amount of 1 mol % to 10 mol % relative to aryl bromide 1.

In a further preferred embodiment of step (a) of the process according to the invention, said palladium catalyst is PdCl₂(dtbpf) and is present in an amount of 1 mol % to 5 mol % relative to aryl bromide 1.

In a further preferred embodiment of step (a) of the process according to the invention, said palladium catalyst is PdCl₂(dtbpf) and is present in an amount of 1 mol % to 3 mol % relative to aryl bromide 1.

In a particularly preferred embodiment of step (a) of the process according to the invention, said palladium catalyst is PdCl₂(dtbpf) and is present in an amount of 2 mol % relative to aryl bromide 1.

In one embodiment of step (a) of the process according to the invention, said base is selected from inorganic bases and alcoholates.

In a preferred embodiment of step (a) of the process according to the invention, said base is selected from Na₂CO₃, Ba(OH)₂, K₃PO₄, Cs₂CO₃, K₂CO₃, TlOH, KF, CsF, Bu₄F, and NaOH.

In a preferred embodiment of step (a) of the process according to the invention, said base is selected from K₃PO₄ and K₂CO₃.

In a particularly preferred embodiment of step (a) of the process according to the invention, said base is K₃PO₄.

In a particularly preferred embodiment of step (a) of the process according to the invention, said base is K₂CO₃.

In one embodiment of step (a) of the process according to the invention 1 to 4 equivalents of said base relative to aryl bromide 1 are used.

In a preferred embodiment of step (a) of the process according to the invention 1 to 3 equivalents of said base relative to aryl bromide 1 are used.

In a further preferred embodiment of step (a) of the process according to the invention 1 to 2 equivalents of said base relative to aryl bromide 1 are used.

In a particularly preferred embodiment of step (a) of the process according to the invention 1.5 equivalents of said base relative to aryl bromide 1 are used.

One advantage of the Suzuki coupling of process step (a) according to the invention is that it performs very well in toluene, as opposed to the toxic dioxane described in WO2019206853. This is of particular importance when conducting the reaction on an industrial scale and in view of the fact that the final antibiotic product of formula (I) is for administration to mammals, where traces of toxic solvents are not acceptable.

Thus, in one embodiment of step (a) of the process according to the invention, step (a) is conducted in an aromatic solvent.

In one embodiment, said aromatic solvent is selected from toluene, xylenes (o-xylene, p-xylene, m-xylene or a mixture thereof), ethylbenzene, anisole, cumene, and cymene.

In a preferred embodiment said aromatic solvent is toluene.

It was found that the methyl ester moiety of boronic acid building block 2 is prone to hydrolysis under the reaction conditions used. However, it was surprisingly found that adding water to the reaction mixture prevents said hydrolysis, allowing to employ fewer equivalents of this building block.

Thus, in one embodiment of step (a) of the process according to the invention, the process is conducted in the further presence of water.

In one embodiment of step (a) of the process according to the invention, the process is conducted in the further presence of 1 to 30 equivalents of water relative to aryl bromide 1.

In one embodiment of step (a) of the process according to the invention, the process is conducted in the further presence of 1 to 20 equivalents of water relative to aryl bromide 1.

In one embodiment of step (a) of the process according to the invention, the process is conducted in the further presence of 5 to 25 equivalents of water relative to aryl bromide 1.

In a preferred embodiment of step (a) of the process according to the invention, the process is conducted in the further presence of 10 to 25 equivalents of water relative to aryl bromide 1.

In a further preferred embodiment of step (a) of the process according to the invention, the process is conducted in the further presence of 12.5 to 25 equivalents of water relative to aryl bromide 1.

In a particularly preferred embodiment of step (a) of the process according to the invention, the process is conducted in the further presence of 25 equivalents of water relative to aryl bromide 1.

In one embodiment of step (a) of the process according to the invention, the process is conducted in the further presence of 5 to 15 equivalents of water relative to aryl bromide 1.

In one embodiment of step (a) of the process according to the invention, the process is conducted in the further presence of 10 to 15 equivalents of water relative to aryl bromide 1.

In a particularly preferred embodiment of step (a) of the process according to the invention, the process is conducted in the further presence of 12.5 equivalents of water relative to aryl bromide 1.

In one embodiment of step (a) of the process according to the invention, the process is conducted in the further presence of water, wherein the water is continuously added over a period of 2-8 h to the reaction mixture comprising aryl bromide 1, boronic acid 2, palladium catalyst and base.

In one embodiment of step (a) of the process according to the invention, the process is conducted in the further presence of water, wherein the water is continuously added over a period of 2-7 h to the reaction mixture comprising aryl bromide 1, boronic acid 2, palladium catalyst and base.

In a preferred embodiment of step (a) of the process according to the invention, the process is conducted in the further presence of water, wherein the water is continuously added over a period of 3-6 h to the reaction mixture comprising aryl bromide 1, boronic acid 2, palladium catalyst and base.

In one embodiment of step (a) of the process according to the invention, the process is conducted in the further presence of water, wherein the water is continuously added over a period of 2-5 h to the reaction mixture comprising aryl bromide 1, boronic acid 2, palladium catalyst and base.

In a preferred embodiment of step (a) of the process according to the invention, the process is conducted in the further presence of water, wherein the water is continuously added over a period of 2-4 h to the reaction mixture comprising aryl bromide 1, boronic acid 2, palladium catalyst and base.

In a particularly preferred embodiment of step (a) of the process according to the invention, the process is conducted in the further presence of water, wherein the water is continuously added over a period of 6 h to the reaction mixture comprising aryl bromide 1, boronic acid 2, palladium catalyst and base.

In a particularly preferred embodiment of step (a) of the process according to the invention, the process is conducted in the further presence of water, wherein the water is continuously added over a period of 3 h to the reaction mixture comprising aryl bromide 1, boronic acid 2, palladium catalyst and base.

As mentioned above, the new Suzuki coupling according to the invention allows the use of less equivalents of boronic acid 2 compared to the amound of boronic acid that is used in the process described in WO2019206853.

Thus, in one embodiment of step (a) of the process according to the invention, 1 to <1.5 equivalents of boronic acid 2 are used relative to aryl bromide 1.

In a preferred embodiment of step (a) of the process according to the invention, 1 to 1.4 equivalents of boronic acid 2 are used relative to aryl bromide 1.

In a further preferred embodiment of step (a) of the process according to the invention, 1 to 1.3 equivalents of boronic acid 2 are used relative to aryl bromide 1.

In a further preferred embodiment of step (a) of the process according to the invention, 1.05 to 1.2 equivalents of boronic acid 2 are used relative to aryl bromide 1.

In a further preferred embodiment of step (a) of the process according to the invention, 1 to 1.2 equivalents of boronic acid 2 are used relative to aryl bromide 1.

In a further preferred embodiment of step (a) of the process according to the invention, 1 to 1.1 equivalents of boronic acid 2 are used relative to aryl bromide 1.

In a particularly preferred embodiment of step (a) of the process according to the invention, 1.2 equivalents of boronic acid 2 are used relative to aryl bromide 1.

In a particularly preferred embodiment of step (a) of the process according to the invention, 1.05 equivalents of boronic acid 2 are used relative to aryl bromide 1.

If desired, the level of palladium contamination of compound 3 may be reduced, typically to a level of <10 ppm, by treating compound 3 with N-acetylcysteine or ammonium pyrrolidinedithiocarbamate in situ.

Thus, in one embodiment, the process for manufacturing compound 3, or a salt thereof, according to the invention optionally further comprises:
(b) adding ammonium pyrrolidinedithiocarbamate or an aqueous solution of N-acetylcysteine to the reaction mixture obtained from step (a).

In one embodiment, the process for manufacturing compound 3, or a salt thereof, according to the invention optionally further comprises:
(b) adding an aqueous solution of N-acetylcysteine to the reaction mixture obtained from step (a).

In one embodiment, the process for manufacturing compound 3, or a salt thereof, according to the invention optionally further comprises:
(b) adding ammonium pyrrolidinedithiocarbamate to the reaction mixture obtained from step (a).

In a further aspect, the present invention provides a process for manufacturing compound 4, or a salt thereof,

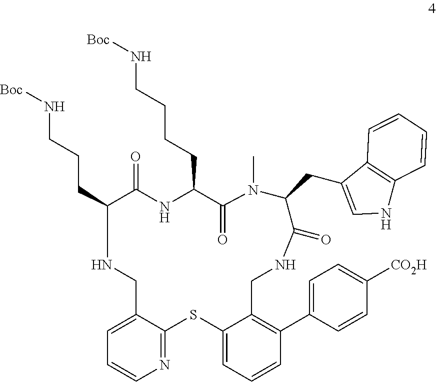

comprising steps (a) and (b) as described herein, and further comprising:
(c) reacting compound 3 described herein with sodium hydroxide to afford said compound 4.

In one embodiment, step (c) is performed in a solvent being an alcohol, preferably in methanol.

In a further aspect, the present invention provides a process for manufacturing compound 4, or a salt thereof, comprising steps (a) and (b) as described herein, and further comprising:
(c) reacting compound 3 according to claim 1 with 2 to 10 equivalents, preferably 3 to 8 equivalents, more preferably 4 to 7 equivalents, in particular 7 equivalents of sodium hydroxide to afford said compound 4, wherein said sodium hydroxide is added as a 5% w/w to 30% w/w, preferably 10% w/w to 20% w/w, in particular a 16% w/w aqueous solution to a solution of compound 3 in an alcoholic solvent or a mixture of water and an alcoholic solvent.

In one embodiment, said alcoholic solvent is methanol.

A major advantage of new compound 4 according to the invention is its crystallinity, allowing it to be purified by crystallization. Especially on an industrial scale, crystallization is a convenient means of purification of chemicals, as opposed to other purification techniques, such as chromatography.

Thus, in one embodiment, the process for manufacturing compound 4 according to the invention optionally further comprises:
(d) crystallizing said compound 4.

In a preferred embodiment of step (d) of the process according to the invention, said crystallizing of said compound 4 is crystallizing of said compound 4 from a mixture of an alcohol and acetone.

In a preferred embodiment, said alcohol is selected from 2-propanol and 1-propanol.

In a particularly preferred embodiment said alcohol is 1-propanol.

In a further aspect, the present invention provides a process for manufacturing the compound of formula (I), or a pharmaceutically acceptable salt thereof,

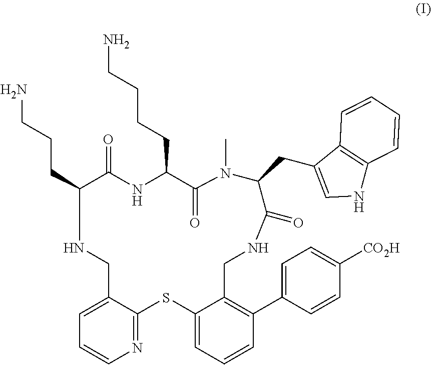

comprising process steps (a)-(d) described herein, and further comprising:
(e) reacting compound 4 described herein with an acid, to afford said compound of formula (I).

In a preferred embodiment of step (e) of the process according to the invention, said acid is hydrochloric acid.

In one embodiment, step (e) is performed in a solvent mixture selected from acetone/water, THF/water, and acetonitrile/water.

In one embodiment, 2 to 6 equivalents of acid relative to compound 4 are used in step (e) of the process according to the invention.

In a preferred embodiment, 3 to 5 equivalents of acid relative to compound 4 are used in step (e) of the process according to the invention.

In a further preferred embodiment, 4 to 5 equivalents of acid relative to compound 4 are used in step (e) of the process according to the invention.

In a further preferred embodiment, 3.5 to 4.5 equivalents of acid relative to compound 4 are used in step (e) of the process according to the invention.

In a particularly preferred embodiment, 4 to 4.1 equivalents of acid relative to compound 4 are used in step (e) of the process according to the invention.

In a further particularly preferred embodiment, 4 equivalents of acid relative to compound 4 are used in step (e) of the process according to the invention.

In a further particularly preferred embodiment, 4.1 equivalents of acid relative to compound 4 are used in step (e) of the process according to the invention.

In one embodiment, step (e) of the process according to the invention is performed between room temperature and reflux.

In a preferred embodiment, step (e) of the process according to the invention is performed between 30 °C and reflux.

In a further preferred embodiment, step (e) of the process according to the invention is performed between 35 °C and 65 °C.

In a particularly preferred embodiment, step (e) of the process according to the invention is performed between 35 °C and 60 °C.

In a further aspect, the present invention provides the use of the process for manufacturing compound 3 described herein in the manufacture of the compound of formula (I).

In a further aspect, the present invention provides the use of the process for manufacturing compound 4 described herein in the manufacture of the compound of formula (I).

### Compounds of the Invention

The present invention relates to certain synthetic intermediates that are useful for manufacturing the novel antibiotic of formula (I) described herein.

The present invention also relates to certain compounds when manufactured according to the novel chemical processes described herein.

In more detail, in one aspect, the present invention provides a compound, which is tert-butyl 3-[[(11S,14S,17S)-14-[4-(tert-butoxycarbonylamino)butyl]-11-[3-(tert-butoxycarbonylamino) propyl]-22-(4-methoxycarbonylphenyl)-16-methyl-12,15,18-trioxo-2-thia-4,10,13,16,19-pentazatricyclo[19.4.0.03,8]pentacosa-1 (25),3,5,7,21,23-hexaen-17-yl]methyl]indole-1-carboxylate (3), or a salt thereof

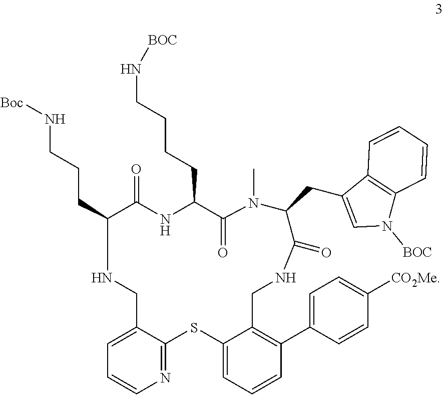

In a further aspect, the present invention provides a compound, which is 4-[(11S,14S,17S)-14-[4-(tert-butoxycarbonylamino)butyl]-11-[3-(tert-butoxycarbonylamino) propyl]-17-(1H-indol-3-ylmethyl)-16-methyl-12,15,18-trioxo-2-thia-4,10,13,16,19-pentazatricyclo[19.4.0.03,8]pentacosa-1 (25),3,5,7,21,23-hexaen-22-yl]benzoic acid (4), or a salt thereof

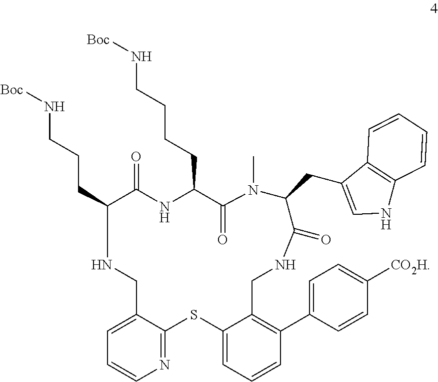

In a further aspect, the present invention relates to compound 3 when manufactured according to the processes for manufacturing compound 3 described herein.

In a further aspect, the present invention relates to compound 3 when manufactured according to the processes for manufacturing compound 3 described herein.

In a further aspect, the present invention relates to the compound of formula (I) when manufactured according to the processes for manufacturing the compound of formula (I) described herein.

In a further aspect, the present invention relates to the use of compound 3 described herein in the manufacture of the compound of formula (I).

In a further aspect, the present invention relates to the use of compound 4 described herein in the manufacture of the compound of formula (I).

### EXAMPLES

The invention will be more fully understood by reference to the following examples. The claims should not, however, be construed as limited to the scope of the examples.

The following abbreviations are used in the present text: PdCl₂(dtbpf)=[1,1′-Bis(di-tert-butylphosphino) ferrocene]dichloropalladium (II) (CAS 95408-45-0); HCl=hydrochloric acid; PPh₃=triphenylphosphine; PtBu₃=tri-tert-butylphosphine; PAd₃=tri (1-adamantyl)phosphine; AmPhos=(4-(N,N-dimethylamino)phenyl)di-tert-butyl phosphine; cataCXiumA=di(1-adamantyl)-n-butylphosphine; PtBu₂Bu=di-t-butyl(n-butyl) phosphine; PtBu₂Ph=di-t-butyl(phenyl) phosphine; PtBu₂-p-C₆H₄CF₃=di-t-butyl (4-trifluoromethyl phenyl)phosphine; cataCXium POMeCy=1-(2-Methoxyphenyl)-2-(dicyclohexylphosphino) pyrrole; CPhos=2-dicyclohexylphosphino-2′,6′-bis(N,N-dimethylamino) biphenyl; RuPhos=2-dicyclohexylphosphino-2′,6′-diisopropoxybiphenyl; SPhos=2-dicyclohexylphosphino-2′,6′-dimethoxybiphenyl; dppf=1,1′-Ferrocenediyl-bis(diphenylphosphine); dcypf=1,1′-Ferrocenediyl-bis(dicyclohexylphosphine); dippf=1,1′-Ferrocenediyl-bis(diisopropylphosphine); dtbpf=1,1′-Bis(di-tert-butylphosphino) ferrocene; Pd(PPh₃)₄=tetrakis(triphenyl-phosphino) palladium; CPhosPdG3=[(2-Dicyclohexylphosphino-2′,6′-bis(N,N-dimethylamino)-1,1′-biphenyl)-2-(2′-amino-1,1′-biphenyl)]palladium (II) methanesulfonate; Pd₂(dba)₃=tris(dibenzylideneacetone) dipalladium (0).

### Example 1

### Preparation of 4-[(11S,14S,17S)-14-[4-(tert-butoxycarbonylamino)butyl]-11-[3-(tert-butoxycarbonylamino) propyl]-17-(1H-indol-3-ylmethyl)-16-methyl-12,15,18-trioxo-2-thia-4,10,13,16,19-pentazatricyclo[19.4.0.03,8]pentacosa-1(25),3,5,7,21,23-hexaen-22-yl]benzoic acid

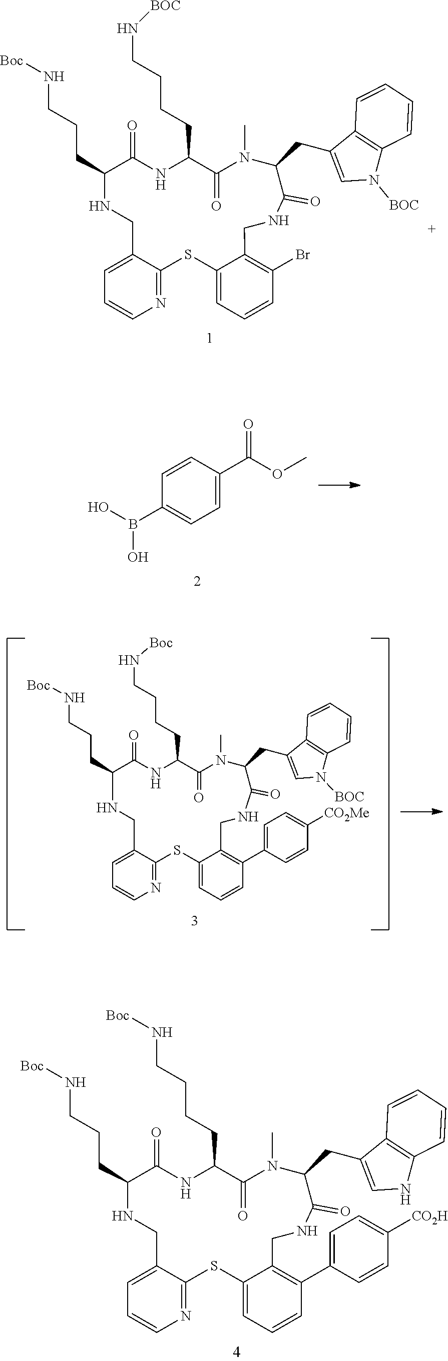

To a dry, Ar-flushed flask were charged 3-[[(11S,14S,17S)-22-bromo-14-[4-(tert-butoxycarbonylamino)butyl]-11-[3-(tert-butoxycarbonylamino) propyl]-12,15,18-triketo-16-methyl-2-thia-4,10, 13, 16, 19-pentazatricyclo[19.4.0.03,8]pentacosa-1 (25),3,5,7,21,23-hexaen-17-yl]methyl]indole-1-carboxylic acid tert-butyl ester (1) (30.0 g, 28.57 mmol, 1.0 eq), tripotassium phosphate (9.10 g, 42.85 mmol, 1.5 eq) and toluene (300 ml, 10 vol). The mixture was degassed and heated to 90 °C. To the hot mixture were added (4-carbomethoxyphenyl) boronic acid (2) (5.4 g, 30.00 mmol, 1.05 eq) and PdCl₂(dtbpf) (372 mg, 0.571 mmol, 0.02 eq). To the resulting suspension water (6.43 g, 357.1 mmol, 12.5 eq) was charged over 3 h via syringe pump. The mixture was stirred for 4 h. The mixture was cooled to 60 °C and 9% w/w aq. N-acetylcysteine was added. The mixture was stirred for 1 h at 60 °C, then the phases were split. The organic phase was extracted with water. The organic layer was treated with activated charcoal at room temperature.

The mixture was distilled under reduced pressure an the solvent was swapped to methanol and concentrated to a final volume of 80 ml. Methanol (221 ml, 7.4 vol) and water (70 ml, 2.3 vol) are added. The mixture is heated to 50 °C and 32% w/w aq. sodium hydroxide (25.0 g, 200.0 mmol, 7.0 eq) is added. The mixture is stirred for 5 h and then cooled to 20 °C. Methanol is distilled off under reduced pressure. Ethyl acetate (250 ml, 8.3 vol) and a solution of citric acid (27.8 g) in water (60 ml, 2 vol) are charged to the residue at room temperature. The organic phase is extracted with a solution of sodium bicarbonate (4.88 g) in water (150 ml) and then with water (150 ml). The organic phase is concentrated to 3 vol under reduced pressure. The solvent is switched to 1-propanol to a final volume of ca. 3.3 vol. The residue is heated to 60 °C. Acetone (198 g, 250 ml, 8.33 vol) is added in 1.5h. The resulting suspension is cooled to 20 °C in 5 h. The solids are recovered by suction filtration and rinsed with a solution of 1-propanol (20 g) and acetone (60 g) and dried under reduced pressure. 23.1 g (81.4% yield) of crude 4-[(11S,14S,17S)-14-[4-(tert-butoxycarbonylamino)butyl]-11-[3-(tert-butoxycarbonylamino) propyl]-17-(1H-indol-3-ylmethyl)-12,15,18-triketo-16-methyl-2-thia-4,10,13,16,19-pentazatricyclo[19.4.0.03,8]pentacosa-1 (25),3,5,7,21,23-hexaen-22-yl]benzoic acid (4) as off-white solid is obtained.

### Example 1a

### Preparation of 4-[(11S,14S,17S)-14-[4-(tert-butoxycarbonylamino)butyl]-11-[3-(tert-butoxycarbonylamino) propyl]-17-(1H-indol-3-ylmethyl)-16-methyl-12,15,18-trioxo-2-thia-4,10,13,16,19-pentazatricyclo[19.4.0.03,8]pentacosa-1 (25),3,5,7,21,23-hexaen-22-yl]benzoic acid

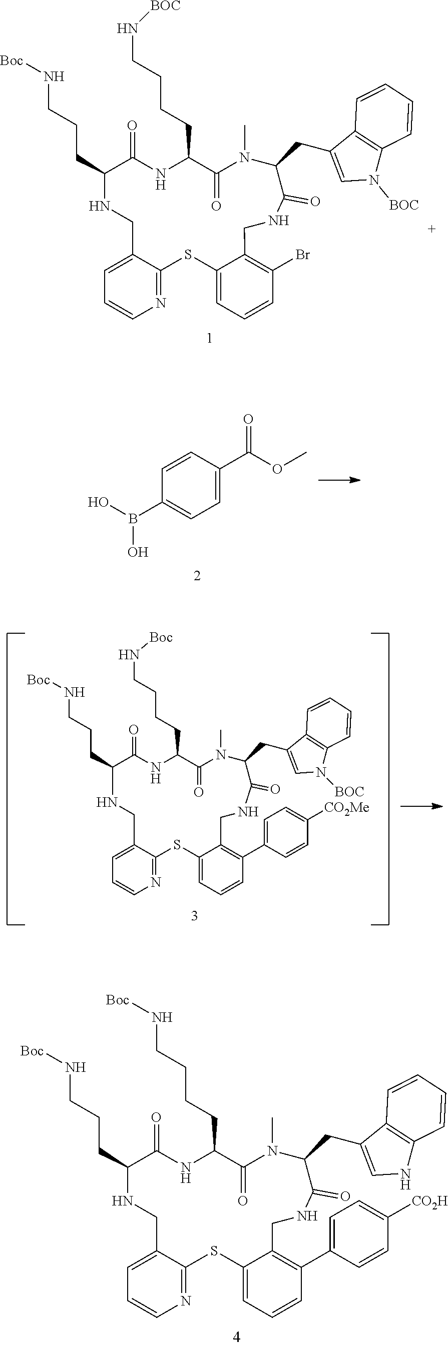

To a dry, Ar-flushed flask were charged 3-[[(11S,14S,17S)-22-bromo-14-[4-(tert-butoxycarbonylamino)butyl]-11-[3-(tert-butoxycarbonylamino) propyl]-12,15,18-triketo-16-methyl-2-thia-4,10, 13, 16, 19-pentazatricyclo[19.4.0.03,8]pentacosa-1 (25),3,5,7,21,23-hexaen-17-yl]methyl]indole-1-carboxylic acid tert-butyl ester (1) (5.0 g, 4.76 mmol, 1.0 eq), potassium carbonate (0.987 g, 7.14 mmol, 1.5 eq) and toluene (50 ml, 10 vol). The mixture was degassed and heated to 90 °C. To the hot mixture were added (4-carbomethoxyphenyl) boronic acid (2) (1.0 g, 5.56 mmol, 1.2 eq) and toluene (25 ml, 5 vol). The mixture is distilled under reduced pressure, until 25 ml distillate are collected. PdCl₂(dtbpf) (62 mg, 0.095 mmol, 0.02 eq) is added and the mixture is stirred ca. 30 min at 85-90 °C. Water (2.14 g, 119.03 mmol, 25.0 eq) was charged over 6 h via syringe pump. The mixture was stirred for at least 2h.

Ammonium pyrrolidinedithiocarbamate (237 mg, 1.43 mmol, 0.3 eq) is added. The mixture was stirred for at least 1 h at 85-90 °C, then the phases were split. The organic phase was extracted with water.

The organic layer was distilled under reduced pressure and methanol (12.5 ml, 2.5 vol) is added. The mixture is cooled to 40 °C. A 16% w/w aqueous soludion of sodium hydroxide (8.33 g, 33.33 mmol, 7.0 eq) is added. The mixture is stirred for at least 5 h and then cooled to 20 °C. A 35% w/w aqueous solution of citric acid (13.07 g, 23.81 mmol, 5.0 eq) is charged to the residue at 20 °C and the mixture is distilled under reduced pressure. The residue is extracted with ethyl acetate. The organic phase is extracted with water, dried over sodium sulfate and diluted with ethyl acetate (15 ml). The solution is heated to 60 °C and stirred for ca. 1 h. The resulting suspension is cooled in 12 h to 0 °C and stirred at least 2 h. The solids are recovered by suction filtration and rinsed with cold ethyl acetate. 3.2 g (80.4% yield) of 4-[(11S,14S, 17S)-14-[4-(tert-butoxycarbonylamino)butyl]-11-[3-(tert-butoxycarbonylamino) propyl]-17-(1H-indol-3-ylmethyl)-16-methyl-12,15,18-trioxo-2-thia-4,10, 13, 16, 19-pentazatricyclo[19.4.0.03,8]pentacosa-1 (25),3,5,7,21,23-hexaen-22-yl]benzoic acid (4) as off-white solid is obtained.

### Example 2

### Preparation of 4-[(11S,14S,17S)-14-(4-aminobutyl)-11-(3-aminopropyl)-17-(1H-indol-3-ylmethyl)-16-methyl-12,15,18-trioxo-2-thia-4,10, 13, 16, 19-pentazatricyclo[19.4.0.03,8]pentacosa-1 (25), 3,5,7,21,23-hexaen-22-yl]benzoic acid

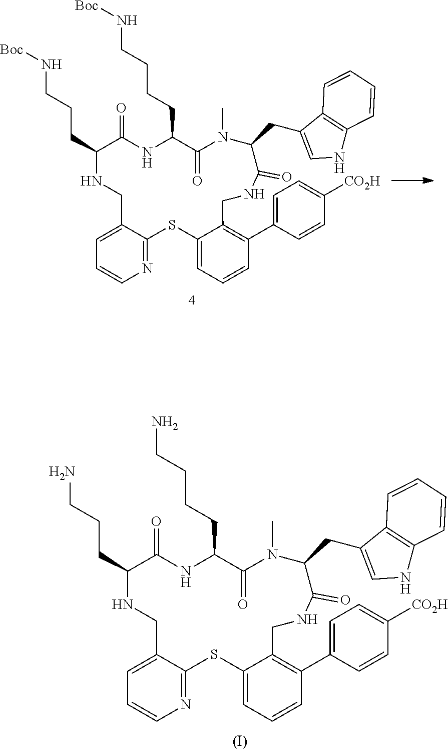

4-[(11S,14S, 17S)-14-[4-(tert-butoxycarbonylamino)butyl]-11-[3-(tert-butoxycarbonylamino) propyl]-17-(1H-indol-3-ylmethyl)-12,15,18-triketo-16-methyl-2-thia-4,10, 13, 16, 19-pentazatricyclo[19.4.0.03,8]pentacosa-1 (25),3,5,7,21,23-hexaen-22-yl]benzoic acid (4) (200 g, 201.78 mmol, 1 eq), tetrahydrofuran (455 ml), and water (320 mL) were charged into a 2000 mL four-necked reaction vessel and stirred at 35 °C. A solution of HCl 25% (99.8 mL, 821 mmol, 4.1 eq.) and water (200 mL) was added within 10 minutes at 35 to 50 °C. The addition funnel was rinsed with water (50 mL) and the reaction mixture was stirred at 60 °C for 4 hours. Following basic workup and crystallization at pH 10, the title compound was obtained as a white powder (158.3 g, 98.5%).

1. A compound, which is tert-butyl 3-[[(11S,14S,17S)-14-[4-(tert-butoxycarbonylamino)butyl]-11-[3-(tert-butoxycarbonylamino) propyl]-22-(4-methoxycarbonylphenyl)-16-methyl-12,15,18-trioxo-2-thia-4,10,13,16,19-pentazatricyclo[19.4.0.03,8]pentacosa-1 (25),3,5,7,21,23-hexaen-17-yl]methyl]indole-1-carboxylate 3, or a salt thereof

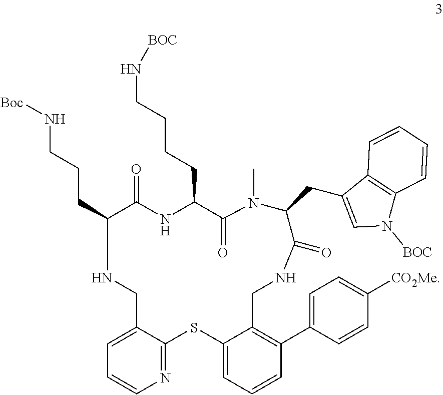

2. A compound, which is 4-[(11S,14S,17S)-14-[4-(tert-butoxycarbonylamino)butyl]-11-[3-(tert-butoxycarbonylamino) propyl]-17-(1H-indol-3-ylmethyl)-16-methyl-12,15,18-trioxo-2-thia-4,10,13,16,19-pentazatricyclo[19.4.0.03,8]pentacosa-1 (25),3,5,7,21,23-hexaen-22-yl]benzoic acid 4, or a salt thereof

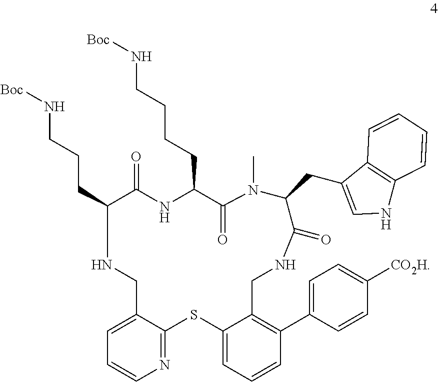

3. A process for manufacturing compound 3 according to claim 1, or a salt thereof, comprising:
(a) reacting aryl bromide (1) 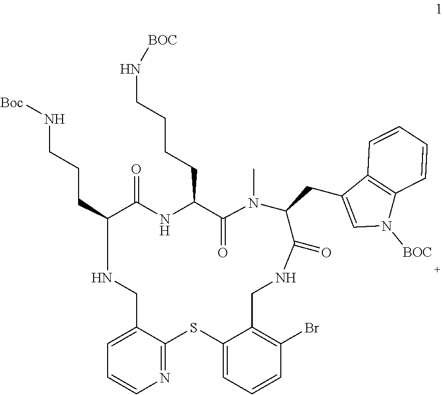
with (4-carbomethoxyphenyl) boronic acid 2 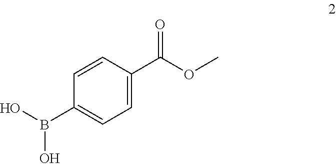
in the presence of a palladium catalyst and a base in an aromatic solvent, to afford said compound 3; wherein
said palladium catalyst is selected from:
i) a preformed catalyst selected from Pd(PPh₃)₄, Pd(L¹)₂, [Pd(L¹)XCl], [Pd(L¹)X]trifluoromethanesulfonate, [Pd(L¹)(2-(2′-amino-1,1′-biphenyl)Cl]; [Pd(L¹)(2-(2′-amino-1,1′-biphenyl)]methanesulfonate, [Pd(L¹)(2-(2′-methylamino-1,1′-biphenyl)]methanesulfonate, [Pd(L¹)(2-(2-aminoethyl)phenyl)Cl], Pd(L²)Cl₂, Pd(L²)₂Cl₂, and [PdI(L³)]₂; wherein
X is selected from allyl, 2-butenyl, 2-methylallyl, 1-phenylallyl, and p-tert-butylindenyl;
L¹ is selected from PBu₃, AmPhos, CPhos, RuPhos, and SPhos;
L² is selected from PtBu₃, AmPhos, PtBu₂Bu, PtBu₂Ph, PtBu₂-p-C₆H₄CF₃)₂, dppf, dcypf, dippf, and dtbpf; and
L³ is selected from PtBu₃, and AmPhos; and
ii) a catalyst that is formed in situ from:
iia) a palladium source selected from palladium bis(dibenzylideneacetone)(Pd(dba)₂), (Pd₂(dba)₃), bis(triphenylphosphine) palladium (II) dichloride (Pd(PPh₃)₂Cl₂), palladium acetate (Pd(OAc)₂, palladium trifluoracetate (Pd(TFA)₂), palladium chloride (PdCl₂), palladium bromide (PdBr₂),palladium iodide (PdI₂), palladium bis acetylacetonate (Pd(acac)₂), (Pd(PPh₃)₄), bis(acetonitrile)-palladium (II) dichloride (PdCl₂ (CH₃CN)₂), cyclopentadienyl allyl palladium, allylpalladium (II) chloride dimer (Pd(allyl)Cl)₂), (2-butenyl) chloropalladium dimer, (2-methylallyl) palladium (II) chloride dimer, palladium (1-phenylallyl) chloride dimer, (p-tert-butylindenyl) palladium (II) chloride dimer, di-μ-chlorobis [2′-(amino-N) [1,1′-biphenyl]-2-yl-C]dipalladium(II), di-μ-mesylbis [2′-(amino-N) [1,1′-biphenyl]-2-yl-C]dipalladium(II), di-μ-mesylbis [2′-(methylamino-N) [1,1′-biphenyl]-2-yl-C]dipalladium(II), and di-u-chlorobis [2-[(dimethylamino)methyl]phenyl-C,N]dipalladium(II); and
iib) a ligand selected from mono- and diphosphines; and wherein
said base is selected from inorganic bases and alcoholates.

4. The process according to claim 3, wherein said palladium catalyst is present in an amount of 1 mol % to 10 mol % relative to aryl bromide 1.

5. The process according to claim 3, wherein 1 to 4 equivalents of said base relative to aryl bromide 1 are used.

6. The process according to claim 3, wherein said aromatic solvent is selected from toluene, xylenes (o-xylene, p-xylene, m-xylene or a mixture thereof), ethylbenzene, anisole, cumene, and cymene.

7. The process according to claim 6, wherein said aromatic solvent is toluene.

8. The process according to claim 3, wherein the process is conducted in the further presence of water.

9. The process according to claim 8, wherein the water is continuously added over a period of 2-8 h to the reaction mixture comprising aryl bromide 1, boronic acid 2, palladium catalyst and base.

10. The process according to claim 3, wherein 1 to <1.5 equivalents of boronic acid 2 are used relative to aryl bromide 1.

11. The process according to claim 3, optionally further comprising:
(b) adding ammonium pyrrolidinedithiocarbamate or an aqueous solution of N-acetylcysteine to the reaction mixture obtained from step (a).

12. The process according claim 3, said process further comprising a process for manufacturing compound 4 according to claim 2, or a salt thereof, comprising:
(c) reacting compound 3 according to claim 1 with 2 to 10 equivalents of sodium hydroxide to afford said compound 4, wherein said sodium hydroxide is added as a 5% w/w to 30% w/w aqueous solution to a solution of compound 3 in an alcoholic solvent or a mixture of water and an alcoholic solvent.

13. The process according to claim 12, wherein said alcoholic solvent in step (c) is methanol.

14. The process according to claim 12, optionally further comprising:
(d) crystallizing said compound 4, preferably from a mixture of 1-propanol and acetone.

15. The process according to claim 14, further comprising a process for manufacturing the compound of formula (I), or a pharmaceutically acceptable salt thereof, 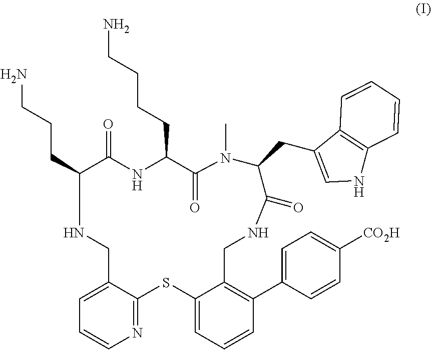
comprising:
(e) reacting compound 4 according to claim 2 with an acid to afford said compound of formula (I).

16. The process according to claim 15, wherein step (e) is performed in a solvent mixture selected from acetone/water, THF/water, and acetonitrile/water.

17. The process according to claim 15, wherein 2 to 6 equivalents of acid relative to compound 4 are used in step (e).

18. The process according to claim 15, wherein the process is performed between room temperature and reflux.

19-25. (canceled)

26. The process according to claim 3, wherein the ligand is selected from PtBu₃, PAd₃, AmPhos, cataCXiumA, PtBu₂Bu, PtBu₂Ph, PtBu₂-p-C₆H₄CF₃, cataCXium POMeCy, CPhos, RuPhos, SPhos, dppf, dcypf, dippf, and dtbpf.

27. The process according to claim 3, wherein the base is selected from Na₂CO₃, Ba(OH)₂, K₃PO₄, Cs₂CO₃, K₂CO₃, TlOH, KF, CsF, Bu₄F, and NaOH.

28. The process according to claim 27, wherein the base is K₃PO₄ or K₂CO₃.

29. The process according to claim 4, wherein said palladium catalyst is present in an amount of 2 mol % relative to aryl bromide 1.

30. The process according to claim 5, wherein 1.5 equivalents of said base relative to aryl bromide 1 are used.

31. The process according to claim 8, wherein the process is conducted in the further presence of 1 to 30 equivalents of water relative to aryl bromide 1.

32. The process according to claim 8, wherein the process is conducted in the further presence of 12.5 to 25 equivalents of water relative to aryl bromide 1.

33. The process according to claim 9, wherein the water is continuously added over a period of 3-6 h to the reaction mixture comprising aryl bromide 1, boronic acid 2, palladium catalyst and base.

34. The process according to claim 10, wherein 1.05 to 1.2 equivalents of boronic acid 2 are used relative to aryl bromide 1.

35. The process according to claim 12, wherein compound 3 is reacted with 7 equivalents of sodium hydroxide in step (c) to afford compound 4.

36. The process according to claim 12, wherein the sodium hydroxide of step (c) is added as a 16% w/w aqueous solution to a solution of compound 3 in an alcoholic solvent or a mixture of water and an alcoholic solvent.

37. The process according to claim 15, wherein compound 4 is reacted with hydrochloric acid in step (e) to afford said compound of formula (I).

38. The process according to claim 17, wherein 4.1 equivalents of acid relative to compound 4 are used in step (e).

39. The process according to claim 18, wherein the process is performed between 35 °C and 60 °C.
